(19) United States
(12) Patent Application Publication
(10) Pub. No.: US 2026/0215848 A1
(43) Pub. Date: Jul. 30, 2026

(54) LASER EMISSION MODULATION FOR TREATMENT OF SOFT TISSUE

(71) Applicant: IPG PHOTONICS CORPORATION, Marlborough, MA (US)

(72) Inventors: Gregory ALTSHULER, Lincoln, MA (US); ILYA YAROSLAVSKY, North Andover, MA (US); James CHILDS, Bolton, MA (US); Anastasia KOVALENKO, Wellesley, MA (US); Dilip PAITHANKAR, Waltham, MA (US)

(73) Assignee: IPG PHOTONICS CORPORATION, Marlborough, MA (US)

(21) Appl. No.: 19/149,148

(22) PCT Filed: Jan. 19, 2024
(86) PCT No.: PCT/US2024/012102

§ 371 Date: Jul. 18, 2025

Related U.S. Application Data
(60) Provisional application No. 63/440,149, filed on Jan. 20, 2023.

(51) Int. Cl. A61B 18/20; A61B 18/00

(52) U.S. Cl. CPC A61B 18/20; A61B 2018/00547; A61B 2018/00577; A61B 2018/00589; A61B 2018/00601; A61B 2018/00607; A61B 2018/00625; A61B 2018/00702; A61B 2018/00761

(57) ABSTRACT

Systems and methods can be used for treating soft tissue. The system can include a laser configured to emit pulsed laser energy that can be modulated to maximize the ablation efficiency during the cutting, incision, or excision of soft tissue while at the same time optimizing hemostasis and reducing collateral side effects such as tissue charring and scar formation. According to certain aspects, a coagulative laser mode power is adjusted mainly for hemostasis without dehydration, and an ablative laser mode power is optimized for providing efficient ablation.

### RELATED APPLICATIONS

The present application claims priority to U.S. Provisional Application Ser. No. 63/440,149, titled “LASER EMISSION MODULATION FOR TREATMENT OF SOFT TISSUE,” filed on Jan. 20, 2023, the content of which is hereby incorporated by reference in its entirety.

### BACKGROUND

### Technical Field

The technical field relates generally to laser treatment of soft tissue, and more specifically to modulated pulsed energy in the treatment of soft tissue.

### Background Discussion

Besides being used for treatment of hard tissue (e.g., kidney stones, bladder stones), laser energy is also widely used to treat various pathologies of soft tissues (e.g., BPH, bladder tumors etc.) through ablation, cutting, vaporization, and coagulation of tissue.

Until recently, relevant laser sources (e.g., Ho:YAG laser) had been extremely limited in their ability to modulate laser emission, i.e., change pulse shape, energy, pulse peak power, and these types of lasers have limited pulse frequencies (pulse repetition rate). However, the advent of the new diode pumped laser sources (e.g., Thulium (Tm) fiber laser, Tm:YAG laser) offer the possibility to modulate the laser across a wider range of output values. There is a need for optimizing the treatment of soft tissue via modulation of the laser emission.

### SUMMARY

Aspects and embodiments are directed to methods and systems of modulating laser emission for treatment of soft tissues using pulsed laser energy.

In accordance with an exemplary embodiment, there is provided a laser system for treating soft tissue that includes a laser configured to emit pulsed laser energy having a wavelength in a range of 1.85 to 2.2 microns (μm) inclusive, and a controller configured to control the laser such that each pulse of the emitted pulse energy comprises two sub-pulses, wherein one sub-pulse of the two sub-pulses is configured to ablate and incise (ablative sub-pulse) a target soft tissue, and the other sub-pulse of the two sub-pulses is configured to coagulate the target soft tissue (hemostatic sub-pulse).

In accordance with another exemplary embodiment, there is provided a method for treating soft tissue that includes generating pulsed laser energy having a wavelength in a range of 1.85 to 2.2 microns (μm) inclusive, emitting each pulse of the emitted pulse energy as two sub-pulses, wherein one sub-pulse of the two sub-pulses is configured to ablate and incise (ablative sub-pulse) a target soft tissue, and the other sub-pulse of the two sub-pulses is configured to coagulate the target soft tissue (hemostatic sub-pulse), and directing the pulsed laser energy at the target soft tissue. In one example, the target soft tissue is hyperplastic prostate tissue.

In one example, the ablative sub-pulse is followed by the hemostatic sub-pulse. In a further example, the ablative sub-pulse and the hemostatic sub-pulse are separated by a sub-pulse interval and the sub-pulse interval is in a range of 0-5 milliseconds (ms) inclusive.

In one example, the hemostatic sub-pulse is followed by the ablative sub-pulse. In a further example, the hemostatic sub-pulse and the ablative sub-pulse are separated by a sub-pulse interval and the sub-pulse interval is in a range of 0 -100 ms inclusive.

In one example, the ablative sub-pulse has a peak power in a range of 400-20,000 Watts (W) inclusive. In another example, the ablative sub-pulse has a peak power in a range of 600-1500 W inclusive. In one example, an energy of the ablative sub-pulse is in a range of 1-10 Joules (J) inclusive. In one example, a duration of the ablative sub-pulse is in a range of 0.05-10 milliseconds (ms) inclusive. In a further example, the duration of the ablative sub-pulse is in a range of 1-10 ms inclusive.

In one example, the target soft tissue has a tissue coagulation threshold and a tissue ablation threshold and the hemostatic sub-pulse has a peak power and an energy that is greater than the tissue coagulation threshold and less than the tissue ablation threshold.

In one example, the target soft tissue has a tissue coagulation threshold and a tissue ablation threshold and the hemostatic sub-pulse has a peak power and an energy that meets or exceeds the tissue coagulation threshold and meets or is less than the tissue ablation threshold multiplied by a factor of 1.5.

In one example, the hemostatic sub-pulse has a peak power in a range of 10-250 W inclusive. In a further example, the hemostatic sub-pulse has a peak power in a range of 50 -150 W inclusive. In one example, an energy of the hemostatic sub-pulse is in a range of 0.5-10 J inclusive. In one example, a duration of the hemostatic sub-pulse is in a range of 2-1000 ms inclusive. In a further example, the duration of the hemostatic sub-pulse configured to coagulate is in a range of 10-100 ms inclusive.

In one example, the ablative sub-pulse is configured to generate a laser-induced bubble in water surrounding the target soft tissue with sufficient pressure to induce mechanical tissue incision on the target soft tissue.

In one example, the laser is a thulium fiber laser, a thulium solid state laser, or a holmium solid state laser.

In accordance with another exemplary embodiment, there is provided a laser system for treating soft tissue that includes a laser configured to emit pulsed laser energy having a wavelength in a range of 1.85 to 2.2 microns (μm) inclusive, and a controller configured to control the laser such that the pulsed laser energy is emitted as a sequence of pulse groups, where each pulse group includes two sub-pulses and the first sub-pulse has a peak power in a range of 10-250 Watts (W) inclusive and a duration of 0.5-100 milliseconds (ms) inclusive, a second sub-pulse has a peak power in a range of 400-20,000 W inclusive and a duration of 0.01-5 ms inclusive, and the pulse groups are separated in time by a group interval that is in a range of 3-250 ms inclusive.

In accordance with another exemplary embodiment, a method for treating a target soft tissue by controlling laser irradiation for preventing soft tissue charring is provided that includes generating pulsed laser energy having a wavelength in a range of 1.85 to 2.2 microns (μm) inclusive, emitting the pulsed laser energy as a sequence of pulse groups, where each pulse group includes two sub-pulses such that the first sub-pulse has a peak power in a range of 10-250 Watts (W) inclusive and a duration of 0.5-100 milliseconds (ms) inclusive, a second sub-pulse has a peak power in a range of 400-20,000 W inclusive and a duration of 0.01-5 ms inclusive, and the pulse groups are separated in time by a group interval that is in a range of 3-250 ms inclusive, and directing the pulsed laser energy at the target soft tissue.

In one example, the group interval is in a range of 3-8 ms inclusive.

In one example, the sub-pulses of the pulse group are separated in time by a sub-pulse interval of a duration to prevent fluid penetration into an area of a target soft tissue that has been exposed to the pulsed laser energy. In another example, the sub-pulse interval is in a range of 0 -30 ms inclusive. In another example, the sub-pulse interval is in a range of 0 -10 ms inclusive. In another example, the sub-pulse interval is in a range of 0-3 ms inclusive.

In one example, the peak power of the second sub-pulse is in a range of 600-1500 W inclusive.

In one example, the peak power of the first sub-pulse is in a range of 50 -250 W inclusive. In a further example, the peak power of the first sub-pulse is in a range of 100-250 W inclusive.

In one example, the second sub-pulse is configured to ablate a charring layer to prevent charring of a target soft tissue being treated by the pulsed laser energy. In another example, the second sub-pulse is configured to prevent charring by ablating any accumulating carbonized tissue layer on the target soft tissue.

In one example, the laser is a thulium fiber laser, a thulium solid state laser, or a holmium solid state laser.

In accordance with another exemplary embodiment, a laser system for treating soft tissue is provided that includes a laser configured to emit pulsed laser energy having a wavelength in a range of 1.85 to 2.2 microns (μm) inclusive, and a controller configured to control the laser such that the pulsed laser energy is emitted as a sequence of pulses where every Nth pulse in the sequence has a peak power in a range of 400-20,000 W inclusive and has a duration of 0.05-5 ms inclusive, and the other pulses in the sequence have a peak power in a range of 10-250 W inclusive and have a duration of 0.5-100 milliseconds (ms) inclusive. In accordance with another exemplary embodiment, a method for treating soft tissue is provided that includes generating pulsed laser energy having a wavelength in a range of 1.85 to 2.2 microns (μm) inclusive, emitting the pulsed laser energy as a sequence of pulses such that every Nth pulse in the sequence has a peak power in a range of 400-20,000 W inclusive and has a duration of 0.05-5 ms inclusive, and the other pulses in the sequence have a peak power in a range of 10-250 W inclusive and have a duration of 0.5-100 milliseconds (ms) inclusive, and directing the pulsed laser energy at a target soft tissue.

In one example, N is from 2-10.

In one example, the Nth pulse is configured to ablate a charring layer to prevent charring of a target soft tissue treated by the pulsed laser energy. In another example, the Nth pulse is configured to prevent charring by ablating any accumulating carbonized tissue layer on the target soft tissue.

In one example, the Nth pulse has a peak power in a range of 600-1500 W inclusive.

In one example, an energy of the Nth pulse is in a range of 0.5-5 Joules (J) inclusive.

In one example, the other pulses in the sequence have a duration of 2-100 ms inclusive.

In one example, the peak power of the other pulses in the sequence is in a range of 50 -250 W inclusive.

In one example, the laser is a thulium fiber laser, a thulium solid state laser, or a holmium solid state laser.

In accordance with another exemplary embodiment, a laser system for treating soft tissue is provided that includes a laser configured to emit laser energy having a wavelength in a range of 1.85 to 2.2 microns (μm) inclusive, and a controller configured to control the laser such that a power of the laser energy is emitted as continuous-wave (CW) laser power with modulation providing overlapping pulses of laser energy.

In accordance with another exemplary embodiment, a method for treating soft tissue is provided that includes generating laser energy having a wavelength in a range of 1.85 to 2.2 microns (μm) inclusive, emitting the laser energy such that a power of the laser energy is emitted as continuous-wave (CW) laser power with modulation providing overlapping pulses of laser energy, and directing the laser energy at a target soft tissue.

In one example, the CW laser power is in a range of 10-250 Watts (W) inclusive and the overlapping pulses each have a peak power in a range of 500-20,000 W inclusive and an average power of the overlapping pulses is in a range of 1-30% of the CW laser power. In another example, the peak power of each overlapping pulse is in a range of 600-1500 W inclusive. In another example, an energy of each overlapping pulse is in a range of 0.5-5 Joules (J) inclusive. In another example, a duration of each overlapping pulse is in a range of 0.05-5 milliseconds (ms) inclusive. In another example, the CW laser power is in a range of 50 -250 W inclusive.

In one example, each pulse of overlapping laser energy is configured to ablate a charring layer to prevent charring of a target soft tissue being treated by the laser energy. In another example, each pulse of overlapping laser energy is configured to prevent charring by ablating any accumulating carbonized tissue layer on the target soft tissue.

In one example, the overlapping pulses are separated in time by a pulse interval that is in a range of 5-200 ms inclusive.

In one example, the overlapping pulses have a repetition rate in a range of 5-200 Hertz (Hz) inclusive. In a further example, the overlapping pulses have a repetition rate in a range of 5-50 Hz inclusive.

In one example, the laser is a thulium fiber laser, a thulium solid state laser, or a holmium solid state laser.

Still other aspects, embodiments, and advantages of these example aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments.

Embodiments disclosed herein may be combined with other embodiments, and references to “an embodiment,” “an example,” “some embodiments,” “some examples,” “an alternate embodiment,” “various embodiments,” “one embodiment,” “at least one embodiment,” “this and other embodiments,” “certain embodiments,” or the like are not necessarily mutually exclusive and are intended to indicate that a particular feature, structure, or characteristic described may be included in at least one embodiment. The appearances of such terms herein are not necessarily all referring to the same embodiment.

### BRIEF DESCRIPTION OF DRAWINGS

Various aspects of at least one embodiment are discussed below with reference to the accompanying figures, which are not intended to be drawn to scale. The figures are included to provide an illustration and a further understanding of the various aspects and embodiments, and are incorporated in and constitute a part of this specification, but are not intended as a definition of the limits of any particular embodiment. The drawings, together with the remainder of the specification, serve to explain principles and operations of the described and claimed aspects and embodiments. In the figures, each identical or nearly identical component that is illustrated in various figures is represented by a like numeral. For purposes of clarity, not every component may be labeled in every figure. In the figures:

FIG. 1 is a chart showing one example of a pulse sequence optimized for soft tissue cutting in accordance with one or more aspects of the invention;

FIG. 2 is a chart showing another example of a pulse sequence optimized for soft tissue cutting in accordance with one or more aspects of the invention;

FIG. 3A is a chart showing yet another example of a pulse sequence optimized for soft tissue cutting in accordance with one or more aspects of the invention;

FIG. 3B is a chart showing yet another example of a pulse sequence optimized for soft tissue cutting in accordance with one or more aspects of the invention;

FIG. 4 is a chart showing yet another example of a pulse sequence optimized for soft tissue cutting in accordance with one or more aspects of the invention;

FIG. 5 is a chart showing yet another example of a pulse sequence optimized for soft tissue cutting in accordance with one or more aspects of the invention; and

FIG. 6 is a block diagram of a laser system for treating soft tissue in accordance with one or more aspects of the invention.

### DETAILED DESCRIPTION

In accordance with one or more embodiments, particular systems and methods of modulating laser emission are considered below for specific purposes of treating soft tissue. As used herein, the term “soft tissue” refers to tissues that connect, support, or surround other structures and organs of the body (human or animal), as well as mucosa, tumors of soft tissue, which can be positioned in anatomical locations of the body (e.g., the bladder, prostate gland, kidney). One or more aspects of this disclosure can be used for diode pumped Tm and Holmium (Ho) doped crystal or fiber lasers with output wavelengths in the range between 1.85 and 2.2 μm inclusive. In accordance with certain embodiments, the methods and systems disclosed herein may be implemented or otherwise provided by a thulium fiber laser, a thulium solid state laser, or a holmium solid state laser.

As mentioned previously, laser energy may be used for the treatment of variety of urological conditions ranging from lithotripsy to soft-tissue surgeries (e.g., benign prostate hyperplasia (BPH) removal as enucleation or vaporization by ablation). The delivery of the laser energy can be optimized through modulation of the laser output, as has been shown in PCT Application No. PCT/US2019/042491, which is published as WO 2020/033121 and is owned by Applicant and incorporated herein by reference in its entirety.

In accordance with one or more embodiments, systems and methods for optimizing laser output are disclosed herein for purposes of:
Maximizing the ablation efficiency during soft tissue cutting/incision/excision,
Optimizing hemostasis,
Improving certain specific surgical procedures, e.g., enucleation of the prostate using laser pulses for separation of hyperplastic tissue from the capsule of the prostate gland, and
Reducing collateral side effects such as tissue charring, scar formation, loss of viable material for biopsy etc.

Furthermore, in accordance with certain embodiments, another objective is to provide a laser system that comprises a laser source and a control unit providing the means to modulate output of the laser source according to the pulsed modulation modes described herein.

In accordance with some embodiments, yet another objective is to provide specific optimal pulse shapes for diode pumped laser systems such as Thulium Fiber Laser (TFL) and Tm:YAG-based (thulium solid-state laser) laser systems, as well as holmium solid state lasers, which may be used in one or more embodiments of this disclosure.

It is to be appreciated that although the primary teachings of this invention target applications of laser energy in urology, applications of these teachings to other medical fields are also within the scope of this disclosure.

### Maximizing Efficiency of Soft Tissue Cutting/Incision/Excision/Nucleation, Optimizing Hemostasis, and Reducing Charring

The successful cutting/incision/excision/nucleation of soft tissue with directed energy (e.g., laser energy) requires the fulfillment of three conditions: 1) efficient ablation that provides a sufficiently deep cut per unit of energy expended; 2) minimum (ideally, absence of) carbonized tissue on the surface of the laser cut (charring); and 3) creation of an optimally deep and uniform coagulation margin, for purposes of ensuring good hemostasis (i.e., prevent and mitigate bleeding, the stoppage of blood flow).

Conventional approaches to soft tissue treatment show that pulsed low peak power (or continuous wave (CW)) treatment results in a sufficient depth of the cut and good hemostasis but creates unacceptable levels of charring. Conventional approaches to soft tissue treatment that use pulsed high peak power result in minimized charring, but the efficiency of ablation is reduced and often leads to non-uniform hemostasis that is insufficient to mitigate bleeding. Therefore, in accordance with at least one embodiment, the objective of the pulse shape optimization is to combine the benefits of the two modes. In certain embodiments, a coagulative laser mode power is adjusted mainly for hemostasis without dehydration, and an ablative laser mode power is optimized for providing efficient ablation.

The target soft tissue treated by certain methods and systems disclosed herein is associated with benign prostate hyperplasia (BPH). In accordance with certain embodiments, the target soft tissue treated by the methods and systems disclosed herein is hyperplastic prostate tissue. A BPH enucleation process involves removing the adenoma of the prostate within the prostatic capsule. A conventional Ho:YAG laser that is configured to perform HoLEP (Ho Laser Enucleation Procedure) produces a high peak power (up to 20 kilowatts (kW)) in a short pulse for a thermo-mechanical mechanism of separation. The process involves separation of adenoma tissue from the capsule of the prostatic gland. This separation can be described as cutting or incision of tissue precisely on a plane of connection between the capsule and adenoma. This mechanism is a combination of creating a mechanical force on the tissue through a laser-induced vaporization bubble that creates a high mechanical pressure on the tissue and simultaneously performing laser ablation and coagulation via a laser pulse propagated through this bubble which has a primarily thermal effect. The disadvantages of this treatment with Ho:YAG include uncontrolled hemostasis and the significant occurrence of bleeding. In contrast, a Thulium Fiber Laser that is configured to perform TFLEP (Thulium Fiber Laser Enucleation Procedure) produces excellent hemostasis, but not as good separation of adenoma tissue within the prostatic capsule. In addition, tissue charring compromises the visibility of the separation plane.

One or more embodiments disclosed herein overcome the problems presented by diode-pumped TFL or Tm:YAG lasers through the combination of two sub-pulses: an ablative sub-pulse with high peak power followed by a coagulative (hemostatic) sub-pulse with low peak power. According to at least one embodiment, one sub-pulse of the two sub-pulses is configured to ablate and incise (referred to as an ablative sub-pulse or ablation sub-pulse) a target soft tissue and the other sub-pulse of the two sub-pulses is configured to coagulate the target soft tissue (referred to as a hemostatic sub-pulse).

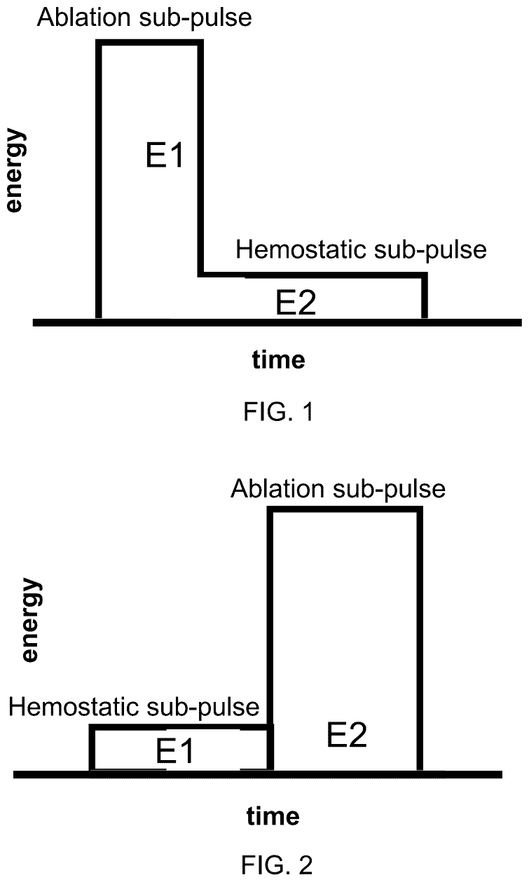

One non-limiting example of such a sub-pulse combination is shown in FIG. 1. In this embodiment, the ablative sub-pulse is followed by the hemostatic sub-pulse (or the ablative sub-pulse is directed at the target soft tissue prior to the hemostatic sub-pulse). In at least one embodiment, the ablative sub-pulse and the hemostatic sub-pulse are separated by a sub-pulse interval and the sub-pulse interval is in a range of 0-5 milliseconds (ms) (inclusive). In the non-limiting example shown in FIG. 1, the ablative sub-pulse and the hemostatic sub-pulse have a sub-pulse interval of 0, meaning they are joined.

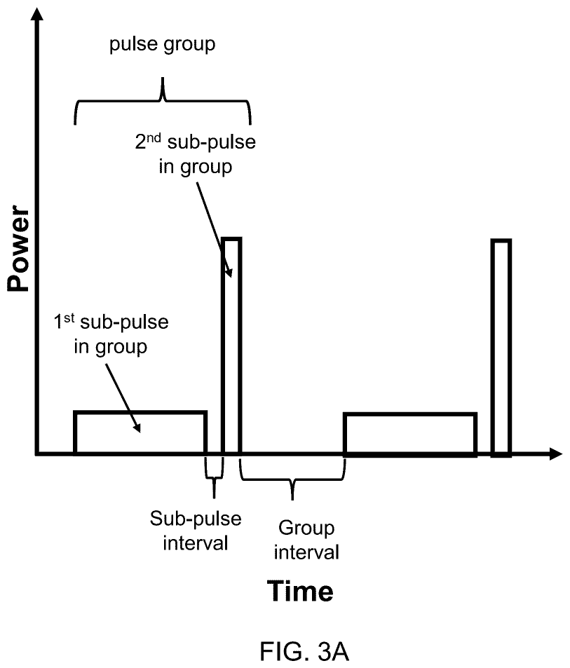

In an alternative embodiment, the hemostatic sub-pulse may precede the ablative sub-pulse (i.e., the hemostatic sub-pulse is followed by the ablative sub-pulse), a non-limiting example of which is shown in FIG. 2. In at least one embodiment, the hemostatic sub-pulse and the ablative sub-pulse are separated by a sub-pulse interval and the sub-pulse interval is in a range of 0 -100 ms (inclusive). In the non-limiting example shown in FIG. 2, the hemostatic sub-pulse and the ablative sub-pulse have a sub-pulse interval of 0, meaning they are joined.

In both scenarios provided above, the following laser operating parameters may be applied, which were arrived at by Applicant after performing pre-clinical and clinical trials:
peak power of the ablative sub-pulse may be in a range of 400 to 20,000 W inclusive, preferably in a range of 600-1500 W inclusive
energy of the ablative sub-pulse may be in a range of 1-10 J inclusive
pulse duration of the ablative sub-pulse may be in a range of 0.05 to 10 ms inclusive, preferably in a range of 1-10 ms inclusive
peak power of the hemostatic sub-pulse may be in a range 10 to 250 W inclusive, preferably in a range of 50 -150 W inclusive
energy of the hemostatic sub-pulse may be in a range of 0.5-10 J inclusive
pulse duration of the hemostatic sub-pulse may be in a range of 2 to 1000 ms inclusive, preferably in a range of 10-100 ms inclusive

In accordance with at least one embodiment and various aspects of the disclosure, the target soft tissue (being treated by the pulsed laser energy) has a tissue coagulation threshold and a tissue ablation threshold and the hemostatic sub-pulse has a peak power and an energy that is greater than the tissue coagulation threshold and less than the tissue ablation threshold. In another embodiment, the hemostatic sub-pulse has a peak power and energy such that these values meet or exceed the tissue coagulation threshold but meet or are less than the tissue ablation threshold multiplied by a factor of 1.5. For example, if for a given set of surgical conditions (e.g., fiber diameter, speed of cutting, gap between tissue and fiber) the tissue coagulation threshold is 50 W and the tissue ablation threshold is 100 W then the peak power of the hemostatic sub-pulse may be in a range of 50 -150 W.

Experiments performed by Applicant have shown that the thermo-mechanical effect generated by such a pulse as described above can be similar to or better than that of a conventional Ho:YAG laser due to the superior thermal ablation effect of the TFL or Tm:YAG laser-induced vaporization bubble. The mechanical effect of this laser-induced bubble can be optimized by taking advantage of the effect of a laser-induced bubble oscillation that occurs between the fiber tip and the tissue, which is a phenomenon discovered by Applicant. This oscillation produces tissues separation using multiple oscillations of positive and negative pressure, which is in contrast to one positive and negative pressure cycle (as in a Ho:YAG procedure). While the phenomenon can be observed for both types of sub-pulses, the positive and negative mechanical pressure amplitude is much higher for the ablative sub-pulse and can be used for better separation of the adenoma tissue from the capsule. In accordance with at least one embodiment, the ablative sub-pulse is configured to generate a laser-induced bubble in water surrounding the target soft tissue with sufficient pressure to induce mechanical tissue incision on the target soft tissue. In another embodiment, the ablative sub-pulse is further configured to apply positive pressure and at least two instances of negative pressure on the target soft tissue.

In accordance with other embodiments, a “cleaning pulse” concept can be implemented for purposes of reducing or otherwise preventing the accumulation of carbon (carbonized tissue layer, i.e., “charring”) during tissue vaporization, cutting, incision, excision, and enucleation. The rationale is that a high peak power cleaning pulse will periodically ablate any accumulating carbon. In at least one embodiment, this can be achieved via the addition of a high peak power cleaning post sub-pulse to each “regular” pulse. For example, in one embodiment, a second sub-pulse (as described in further detail below and is also referred to herein as the cleaning pulse), is configured to ablate a charring layer to prevent charring of a target soft tissue being treated by the pulsed laser energy, and in a further embodiment, the second sub-pulse is configured to prevent charring by ablating any accumulating carbonized tissue layer on the target soft tissue. A carbonized tissue layer or charring layer is formed on the surface of the laser crater or cut during a tissue ablation procedure. After tissue ablation and water vaporization (e.g., 100-300° C. tissue temperature) dried tissue continues to be heated and when the temperature reaches about 150° C., a thermal chemical reaction (pyrolysis) occurs, and tissue proteins release atoms of carbon. This process is completed at temperatures in a range of 250-300° C. with formation of a carbonized layer of tissue having a typical thickness of 50-500 microns (μm) on the surface of the laser cut. The carbonized layer comprises the surgeon's ability to recognize types of tissue and treatment area conditions and slows down the healing process. The thickness of such a layer increases at lower powers and with increasing pulsewidth or dwell time on the tissue. However, lower powers and a longer pulsewidth yield higher ablation efficiencies and a better coagulation margin. Treating with a short pulse and a high peak power yields a lower hemostatic effect but results in better ablation of the thin layer of carbonized dry tissue due to the better matching of the thermal relaxation time of the layer and the high absorption coefficient of the dry carbonized tissue versus non-carbonized dry tissue.

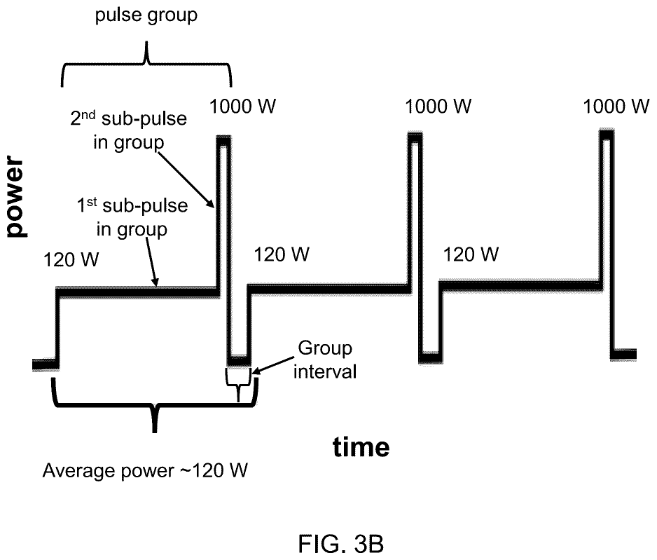

In accordance with certain embodiments, a controller is configured to control the laser such that the pulsed laser energy is emitted as a sequence of pulse groups, where each pulse group includes two sub-pulses. Two non-limiting examples of such a configuration are shown in FIGS. 3A and 3B. In accordance with certain embodiments, the peak power of the cleaning post sub-pulse (second sub-pulse) can be in a range 400 to 20,000 W inclusive, preferably in a range of 600-1500 W inclusive, and has a duration 0.01 to 5 ms inclusive. In certain embodiments, a peak power of the regular portion of the pulse (first sub-pulse) can be in the range 10 to 250 W inclusive, preferably in a range of 50 to 250 W inclusive or in a range of 100-250 W inclusive, and have a duration 0.5 to 100 ms. In some embodiments, the pulse groups are separated in time by a group interval (as shown in FIGS. 3A and 3B) that is in a range of 3-250 ms (inclusive), which corresponds to a repetition rate of the pulse groups that is in a range of 3-300 Hz. In other embodiments, the group interval is in a range of 3-8ms (inclusive), which corresponds to a repetition rate of 10-50 Hz.

Water can penetrate into the laser crater between pulses and additional laser energy is needed to vaporize this water. This particular phenomenon is the reason for significant drops in tissue ablation efficiency and reduced depths of the cut. To prevent this from happening, in accordance with at least one embodiment the sub-pulses of the pulse group are separated in time by a sub-pulse interval of a duration to prevent fluid penetration into an area of a target soft tissue that has been exposed to the pulsed laser energy. In other words, the sub-pulse interval should be shorter than the time that it takes to fill a laser crater (created on the soft tissue by the laser energy) with water (fluid) between the sub-pulses. The duration of the sub-pulse interval varies depending on the depth of ablation. According to some embodiments the sub-pulse interval is in a range of 0 -30 ms (inclusive), in further embodiments the sub-pulse interval is in a range of 0 -10 ms (inclusive), and in further embodiments, the sub-pulse interval is in a range of 0-3 ms (inclusive). The non-limiting example shown in FIG. 3B shows a sub-pulse interval having a value of zero, meaning the first and second sub-pulse are joined.

In accordance with at least one embodiment, the goal of reducing charring via a cleaning pulse technique is achieved through employing an amplitude-modulated (AM) sequence, where every Nth pulse (where N=2-10, i.e., N is a positive integer from 2-10) in the sequence is a high peak power pulse (“cleaning pulse”), whereas the pulses in the rest of the sequence have a low peak power (ablative pulses). For example, in one embodiment, a controller is configured to control the laser such that the pulsed laser energy is emitted as a sequence of pulses where every Nth pulse in the sequence is configured as a cleaning pulse. The Nth pulse is configured to ablate a charring layer to prevent charring of a target soft tissue treated by the pulsed laser energy and in a further embodiment, the Nth pulse is configured to prevent charring by ablating any accumulating carbonized tissue on the target soft tissue, in a similar manner as described previously.

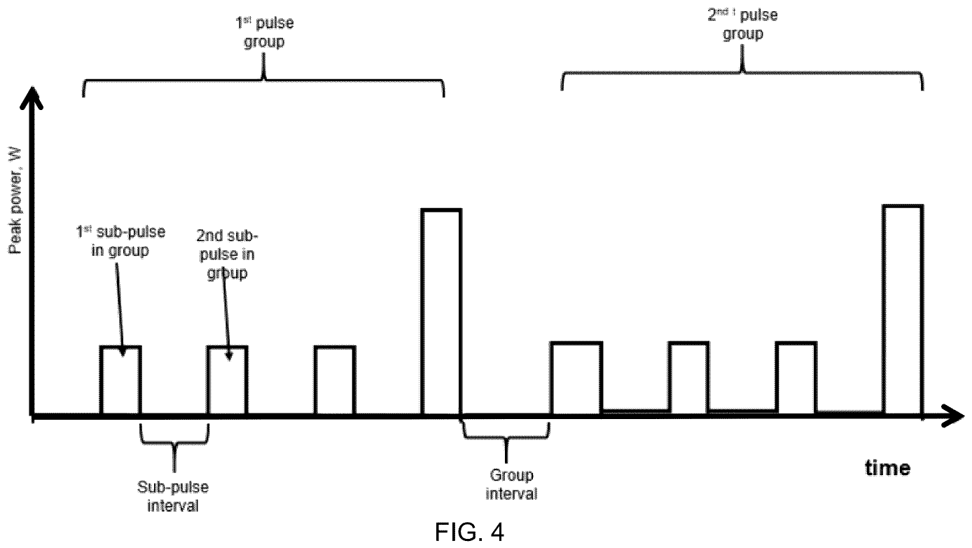

One non-limiting example of such a sequence is shown in FIG. 4, where N=4, so every 4^{th} pulse is a cleaning pulse. In a similar manner as the described previously, the peak power of the cleaning pulse (i.e., every Nth pulse in the sequence) can be in a range of 400 to 20,000 W inclusive, preferably in a range of 600-1500 W inclusive, and has a duration in a range of 0.01 to 5 ms (inclusive). In certain embodiments, the Nth pulse has an energy in a range of 0.5-5 J inclusive. In certain embodiments, a peak power of the regular pulse (the other pulses in the sequence) may be in a range 10 to 250 W inclusive, preferably in a range of 50 -250 W inclusive, and have a duration in a range of 0.5 to 100 ms inclusive, in some embodiments have a duration of 2-100 ms (inclusive), and in other embodiment have a duration in a range of 10-100 ms inclusive. According to some embodiments, a pulse repetition rate can be in a range of 2-1000 Hz (inclusive). In some embodiments, the sequence of pulses may include groups of pulses that are separated by a group interval. For example, in FIG. 4, there are 4 sub-pulses in each pulse group.

In yet another embodiment, a continuous-wave (CW) mode is used and overlapped (overlaid, superimposed) with a sequence of cleaning pulses. In accordance with at least one embodiment, a controller is configured to control the laser such that a power of the laser energy is emitted as continuous-wave laser power with modulation providing overlapping pulses of laser energy.

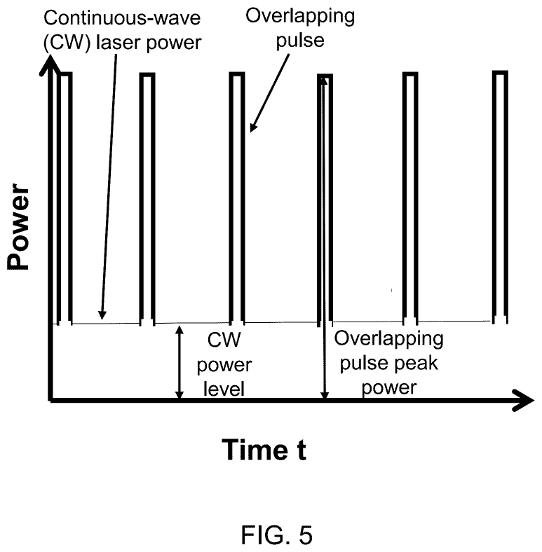

A non-limiting example of such a pulse sequence is shown in FIG. 5. In this configuration, the CW background power (a “baseline” laser emission) can be in a range of 10 to 250 W inclusive, preferably in a range of 50 -250 W inclusive, and is modulated to provide overlapping pulses of laser energy that function as a cleaning pulse. In some embodiments, the overlapping pulses each have a peak power in a range 500 to 20,000 W inclusive, with some embodiments in a range of 500 -1500 W inclusive, and in other embodiment in a range of 600-1500 W inclusive. In accordance with at least one embodiment, an average power of the overlapping pulses is in a range of 1-30% of the CW laser power. For instance, if the CW laser power is 100 W, an average power of the overlapping pulses can be in a range of 1-30 W. In certain embodiments, the cleaning pulse (i.e., the overlapping pulse) has a duration in a range of 0.05 to 5 ms inclusive. In accordance with at least one embodiment, an energy of the overlapping pulses is in a range of 0.5-5 J inclusive. In some embodiments, the repetition rate of the cleaning pulse (overlapping pulse) is in a range of 5-200 Hz inclusive, preferably in a range of 5-50 Hz inclusive. In some embodiments, the overlapping pulses are separated in time by a pulse interval that is in a range of 0.3-10 ms inclusive, and in other embodiments the pulse interval is in a range of 5-200 ms inclusive. The CW-Pulse operational mode described herein has been found by Applicant to be the most efficient regime for deep tissue ablation, cutting, and incision. In one embodiment, each pulse of overlapping laser energy is configured to ablate a charring layer to prevent charring of a target soft tissue treated being treated by the laser energy, and in a further embodiment, each pulse of overlapping laser energy is configured to prevent charring by ablating any accumulating carbonized tissue on the target soft tissue, in a similar manner as described previously.

### Laser System

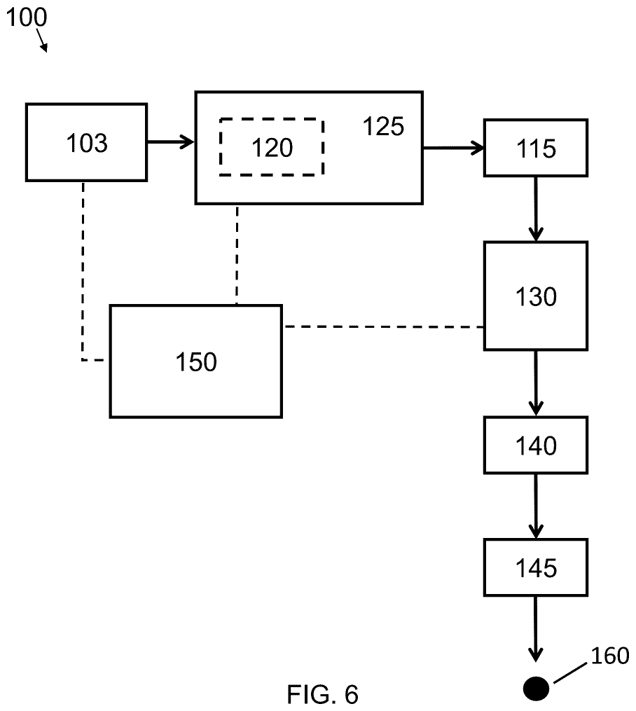

FIG. 6 is a block diagram that shows one non-limiting example of a laser system configured to generate the laser pulse modes described above. It is to be appreciated that other configuration may be used to implement the aforementioned pulse modes.

Laser system 100 comprises a power supply 103, a laser driver 125 that may include an optional energy storage device 120, a pump 115, a laser module 130, and a controller 150, also referred to herein as a control module. Laser energy from the laser module 130 is directed to the target soft tissue 160. Laser system 100 may also include a beam delivery system or module 145 and an optical coupler 140.

The pump 115 is configured with one or more diode lasers that energize laser 130. The power supply 103 supplies power to the system and optional energy storage device 125 (e.g., electrical capacitor and/or inductor) that can be configured to store a sufficient amount of energy necessary to form a laser pulse. The laser driver 125 of the pump 115 forms an electrical pulse of specified characteristics in response to a control signal from control module 150. The electrical pulses are received by one or more diodes of pump 115 which form an optical pulse necessary to pump the laser medium in laser module 130. The output of the laser module 130 is coupled to a beam delivery system 145, and in some instances this coupling occurs via optical coupler 140.

One or more components of the laser system 100 are controlled by controller 150, which is programmed with control signals that are used to control the laser driver 125, power supply 103, and/or laser module 130. For instance, the control signal from controller 150 can be used to directly modulate the pump current of driver 125 used to pump diodes 115, as will be appreciated by those skilled in the art, to output the desired pulse energy, power, and temporal structure. In some embodiments, a single charge-discharge cycle of the energy storage device 120 can be used to modulate the diode current.

The aspects disclosed herein in accordance with the present invention, are not limited in their application to the details of construction and the arrangement of components set forth in the following description or illustrated in the accompanying drawings. These aspects are capable of assuming other embodiments and of being practiced or of being carried out in various ways. Examples of specific implementations are provided herein for illustrative purposes only and are not intended to be limiting. In particular, acts, components, elements, and features discussed in connection with any one or more embodiments are not intended to be excluded from a similar role in any other embodiments.

Also, the phraseology and terminology used herein is for the purpose of description and should not be regarded as limiting. Any references to examples, embodiments, components, elements or acts of the systems and methods herein referred to in the singular may also embrace embodiments including a plurality, and any references in plural to any embodiment, component, element or act herein may also embrace embodiments including only a singularity. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. In addition, in the event of inconsistent usages of terms between this document and documents incorporated herein by reference, the term usage in the incorporated reference is supplementary to that of this document; for irreconcilable inconsistencies, the term usage in this document controls. Moreover, titles or subtitles may be used in the specification for the convenience of a reader, which shall have no influence on the scope of the present invention.

Having thus described several aspects of at least one example, it is to be appreciated that various alterations, modifications, and improvements will readily occur to those skilled in the art. For instance, examples disclosed herein may also be used in other contexts. Such alterations, modifications, and improvements are intended to be part of this disclosure, and are intended to be within the scope of the examples discussed herein. Accordingly, the foregoing description and drawings are by way of example only.

1. A laser system for treating soft tissue, comprising:
a laser configured to emit pulsed laser energy having a wavelength in a range of 1.85 to 2.2 microns (μm) inclusive; and
a controller configured to control the laser such that each pulse of the emitted pulse energy comprises two sub-pulses, wherein
one sub-pulse of the two sub-pulses is configured to ablate and incise (ablative sub-pulse) a target soft tissue, and the other sub-pulse of the two sub-pulses is configured to coagulate the target soft tissue (hemostatic sub-pulse).

2. The laser system of claim 1, wherein th e ablative sub-pulse is followed by the hemostatic sub-pulse.

3. The laser system of claim 2, wherein the ablative sub-pulse and the hemostatic sub-pulse are separated by a sub-pulse interval and the sub-pulse interval is in a range of 0-5 milliseconds (ms) inclusive.

4. The laser system of claim 1, wherein the hemostatic sub-pulse is followed by the ablative sub-pulse.

5. The laser system of claim 4, wherein the hemostatic sub-pulse and the ablative sub-pulse are separated by a sub-pulse interval and the sub-pulse interval is in a range of 0 -100 ms inclusive.

6. The laser system of claim 1, wherein the ablative sub-pulse has a peak power in a range of 400-20,000 Watts (W) inclusive.

7. The laser system of claim 6, wherein the ablative sub-pulse has a peak power in a range of 600-1500 W inclusive.

8. The laser system of claim 1, wherein an energy of the ablative sub-pulse is in a range of 1-10 Joules (D) inclusive.

9. The laser system of claim 1, wherein a duration of the ablative sub-pulse is in a range of 0.05-10 milliseconds (ms) inclusive.

10. The laser system of claim 9, wherein the duration of the ablative sub-pulse is in a range of 1-10 ms inclusive.

11. The laser system of claim 1, wherein the target soft tissue has a tissue coagulation threshold and a tissue ablation threshold and the hemostatic sub-pulse has a peak power and an energy that is greater than the tissue coagulation threshold and less than the tissue ablation threshold.

12. The laser system of claim 1, wherein the soft tissue has a tissue coagulation threshold and a tissue ablation threshold and the hemostatic sub-pulse has a peak power and an energy that meets or exceeds the tissue coagulation threshold and meets or is less than the tissue ablation threshold multiplied by a factor of 1.5.

13. The laser system of claim 1, wherein the hemostatic sub-pulse has a peak power in a range of 10-250 W inclusive.

14. The laser system of claim 13, where in the hemostatic sub-pulse has a peak power in a range of 50 -150 W inclusive.

15. The laser system of claim 1, wherein energy of the hemostatic sub-pulse is in a range of 0.5-10 J inclusive.

16. The laser system of claim 1, wherein a duration of the hemostatic sub-pulse is in a range of 2-1000 ms inclusive.

17. The laser system of claim 16, wherein the duration of the hemostatic sub-pulse configured to coagulate is in a range of 10-100 ms Inclusive.

18. The laser system of claim 1, wherein the ablative sub-pulse is configured to generate a laser-induced bubble in water surrounding the target soft tissue with sufficient pressure to induce mechanical tissue incision on the ta get soft tissue.

19. The laser system of claim 1, wherein the laser is a thulium fiber laser, a thulium solid state laser, or a holmium solid state laser.

20-105. (canceled)
